(19) United States
(12) Patent Application Publication
(10) Pub. No.: US 2013/0046326 A1
(43) Pub. Date: Feb. 21, 2013

(54) METHODS AND SYSTEMS FOR PERFORMING INTRALUMENAL PROCEDURES

(71) Applicants: Donald K. Jones, Dripping Springs, TX (US); Vladimir Mitelberg, Austin, TX (US)

(72) Inventors: Donald K. Jones, Dripping Springs, TX (US); Vladimir Mitelberg, Austin, TX (US)

(21) Appl. No.: 13/589,125

(22) Filed: Aug. 18, 2012

Related U.S. Application Data
(60) Provisional application No. 61/525,349, filed on Aug. 19, 2011.

(51) Int. Cl. A61M 29/02

(52) U.S. Cl. 606/195

(57) ABSTRACT

Devices, systems and methods are provided for performing implantation procedures in a desired area of the body. Systems include embodiments of medical implants that include scaffold and inflatable portions and delivery systems to position and release the medical implants at a target location within the body.

### CROSS-REFERENCE TO RELATED APPLICATIONS

This application claims the benefit of U.S. Prov. Ser. 61/525,349 filed Aug. 19, 2011 which is hereby incorporated by reference herein in its entirety.

### BACKGROUND OF THE INVENTION

The field of intralumenal therapy for the treatment of vascular disease states has for many years focused on the use of many different types of therapeutic devices. While it is currently unforeseeable that one particular device will be suitable to treat all types of vascular disease states it may however be possible to reduce the number of devices used for some disease states while at the same time improve patient outcomes at a reduced cost. To identify potential opportunities to improve the efficiency and efficacy of the devices and procedures it is important for one to understand the state of the art relative to some of the more common disease states.

For instance, one aspect of cerebrovascular disease in which the wall of a blood vessel becomes weakened. Under cerebral flow conditions the weakened vessel wall forms a bulge or aneurysm which can lead to symptomatic neurological deficits or ultimately a hemorrhagic stroke when ruptured. Once diagnosed a small number of these aneurysms are treatable from an endovascular approach using various embolization devices. These embolization devices include detachable balloons, coils, polymerizing liquids, gels, foams, stents and combinations thereof.

Detachable balloons were some of the earliest embolization devices used to treat aneurysms. Under fluoroscopic guidance these balloons were positioned within the aneurysm, inflated using a radio-opaque fluid and subsequently detached from their delivery mechanism. There were numerous drawbacks encountered while using these devices such as difficulty in guiding the devices to the treatment site due to size and shape, difficulties in placing the devices within the aneurysm due to the geometry of the balloons relative to the aneurysm geometry, excessive forces generated during detachment the balloons from the delivery system, dislodging of previously place balloons and delayed deflation of the detached balloons. Examples of various detachable balloon systems attempting to address some of the aforementioned drawbacks are disclosed in U.S. Pat. No. 3,834,394 to Hunter entitled, “Occlusion Device and Method and Apparatus for Inserting the Same”, U.S. Pat. No. 4,085,757 to Pevsner entitled, “Miniature Balloon Catheter Method and Apparatus, U.S. Pat No. 4,327,734 to White Jr. entitled, “Therapeutic Method of Use for Miniature Detachable Balloon” U.S. Pat No. 4,364,392 to Strother entitled “Detachable Balloon Catheter”, U.S. Pat. No. 4,402,319 to Handa, entitled, “Releasable Balloon Catheter”, U.S. Pat. No. 4,517,979 to Pecenka, entitled, “Detachable Balloon Catheter”, U.S. Pat. No. 4,545,367 to Tucci entitled, “Detachable Balloon Catheter and Method of Use”, U.S. Pat. No. 5,041,090 to Scheglov entitled, “Occluding Device” and U.S. Pat. No. 6,379,329 to Naglreiter entitled, “Detachable Balloon Embolization Device and Method.” Although the presented detachable balloon systems and improvements are numerous, few have been realized as commercial products for aneurysm treatment largely due to an inability to address a majority of the previously mentioned drawbacks.

The most widely used embolization devices are detachable embolization coils. These coils are generally made from biologically inert platinum alloys. To treat an aneurysm, the coils are navigated to the treatment site under fluoroscopic visualization and carefully positioned within the dome of an aneurysm using sophisticated, expensive delivery systems. Typical procedures require the positioning and deployment of multiple embolization coils which are then packed to a sufficient density as to provide a mechanical impediment to flow impingement on the fragile diseased vessel wall. Some of these bare embolization coil systems have been describe in U.S. Pat. No. 5,108,407 to Geremia, et al., entitled, “Method And Apparatus For Placement Of An Embolic Coil” and U.S. Pat. No. 5,122,136 to Guglielmi, et al., entitled, “Endovascular Electrolytically Detachable Guidewire Tip For The Electroformation Of Thrombus In Arteries, Veins, Aneurysms, Vascular Malformations And Arteriovenous Fistulas.” These patents disclose devices for delivering embolic coils at predetermined positions within vessels of the human body in order to treat aneurysms, or alternatively, to occlude the blood vessel at a particular location. Many of these systems, depending on the particular location and geometry of the aneurysm, have been used to treat aneurysms with various levels of success. One drawback associated with the use of bare embolization coils relates to the inability to adequately pack or fill the aneurysm due to the geometry of the coils which can lead to long term recanalization of the aneurysm with increased risk of rupture.

Some improvements to bare embolization coils have included the incorporation of expandable foams, bioactive materials and hydrogel technology as described in the following U.S. Pat. No. 6,723,108 to Jones, et al., entitled, “Foam Matrix Embolization Device”, U.S. Pat. No. 6,423,085 to Murayama, et al., entitled, “Biodegradable Polymer Coils for Intraluminal Implants” and U.S. Pat. No. 6,238,403 to Greene, et al., entitled, “Filamentous Embolic Device with Expansible Elements.” While some of these improved embolization coils have been moderately successful in preventing or reducing the rupture and re-rupture rate of some aneurysms, the devices have their own drawbacks. For instance, in the case of bioactive coils, the materials eliciting the biological healing response are somewhat difficult to integrate with the coil structure or have mechanical properties incompatible with those of the coil making the devices difficult to accurately position within the aneurysm. In the case of some expandable foam and hydrogel technology, the expansion of the foam or hydrogel is accomplished due to an interaction of the foam or hydrogel with the surrounding blood environment. This expansion may be immediate or time delayed but is generally, at some point, out of the control of the physician. With a time delayed response the physician may find that coils which were initially placed accurately and detached become dislodged during the expansion process leading to subsequent complications.

For many aneurysms, such as wide necked or fusiform aneurysms the geometry is not suitable for coiling alone. To somewhat expand the use of embolization coils in treating some wide necked aneurysms, stent like scaffolds have been developed to provide support for coils. These types of stent like scaffolds for use in the treatment of aneurysms have been described in U.S. Pat. No. 6,605,111 to Bose et al., entitled, “Endovascular Thin Film Devices and Methods for Treating Strokes” and U.S. Pat. No. 6,673,106 to Mitelberg, et al., entitled, “Intravascular Stent Device”. While these stent like devices have broadened the types of aneurysms amenable to embolization therapy, utilization of these devices in conjunction with embolization devices is technically more complex for the physician, may involve more risk to the patient and have a substantial cost increase for the healthcare system.

To further expand the types of aneurysm suitable for interventional radiological treatment, improved stent like devices have been disclosed in U.S. Pat. No. 5,824,053 to Khosravi et al., entitled, “Helical Mesh Endoprosthesis and Method”, U.S. Pat. No. 5,951,599 to McCrory, entitled, “Occlusion System for the Endovascular Treatment of and Aneurysm” and U.S. Pat. No. 6,063,111 to Hieshima et al., entitled, “Stent Aneurysm Treatment System and Method.” When placed across the neck of an aneurysm the proposed stent like devices purport to have a sufficient density through the wall of the device to reduce flow in the aneurysm allowing the aneurysm to clot, while at the same time having a low enough density through the wall to allow small perforator vessels adjacent to the aneurysm to remain patent. Stent devices of this nature while having the potential to reduce treatment costs have not been realized commercially due to the difficulty in manufacturing, reliability in delivering the devices to the treatment site and an inability to properly position the more dense portion of the stent device accurately over the neck of the aneurysm.

### SUMMARY OF THE INVENTION

The present invention is directed toward a medical implant system for use in placing a medical implant at a preselected site within the body of a mammal. In accordance with one aspect of the present invention there is provided an embolization system for use in a mammal. The embolization system includes an elongate flexible delivery system coupled to an embolization device. The embolization device comprises an elongate embolic coil member coupled to an expandable embolic balloon member. The delivery system includes an elongate tubular filling member positioned within the lumen of an elongate tubular positioning member both having proximal and distal ends and wherein the distal end of the filling member is removably coupled to the embolic balloon member and adapted to provide fluid access to the interior of the balloon member. A valve member (normally biased closed) is included with the balloon member such that when sufficient fluid has been delivered to expand the balloon member to a desired volume, the tubular filling member may be uncoupled from the balloon member thereby allowing the valve member to seal the balloon member and maintain the balloon member inflation.

In accordance with another aspect of the present invention there is provided an embolization device having an elongate scaffold portion and an expandable portion where the expandable portion includes a balloon member and the scaffold portion takes the form of a radiopaque embolic coil.

In accordance with yet another aspect of the present invention there is provided an embolization device having a coating that includes bioactive materials. The bioactive materials may include bioerodible and or biodegradable synthetic materials. The coating may be applied to the scaffold portion and or the expandable portion and further comprise one or more pharmaceutical substances or drug compositions for delivering to the tissues adjacent to the site of implantation, and one or more ligands, such as peptides which bind to cell surface receptors, small and/or large molecules, and/or antibodies or combinations thereof for capturing and immobilizing, in particular progenitor endothelial cells on the blood contacting surface of the device to promote healing.

In accordance with still another aspect of the present invention, there is provided a method of deploying a medical implant within a portion of a vessel. The method comprises the steps of: positioning a catheter adjacent a target site; delivering an embolization system having an embolization device and delivery system to the target site; deploying the embolization device at the target site; inflating the embolization device with a fluid to increase the volume of a portion of the embolization device; releasing the embolization device from the delivery system; sealing the inflated portion of the embolization device; removing the delivery system and catheter from the patient.

### BRIEF DESCRIPTION OF THE DRAWINGS

FIG. 1 is a partially sectioned view of an embodiment of a medical implant system of the present invention.

FIG. 2 is an enlarged partially sectioned view illustrating the distal portion of the medical implant system shown in FIG. 1.

FIG. 3A is a partial cross-sectional view of an embolization device according to an embodiment of the present invention.

FIG. 3B is a partial cross-sectional view of an embolization device according to another embodiment of the present invention.

FIGS. 4 through 8 are partial section views illustrating a method of deploying a medical implant within an aneurysm according to an embodiment of the present invention.

### DETAILED DESCRIPTION OF THE INVENTION

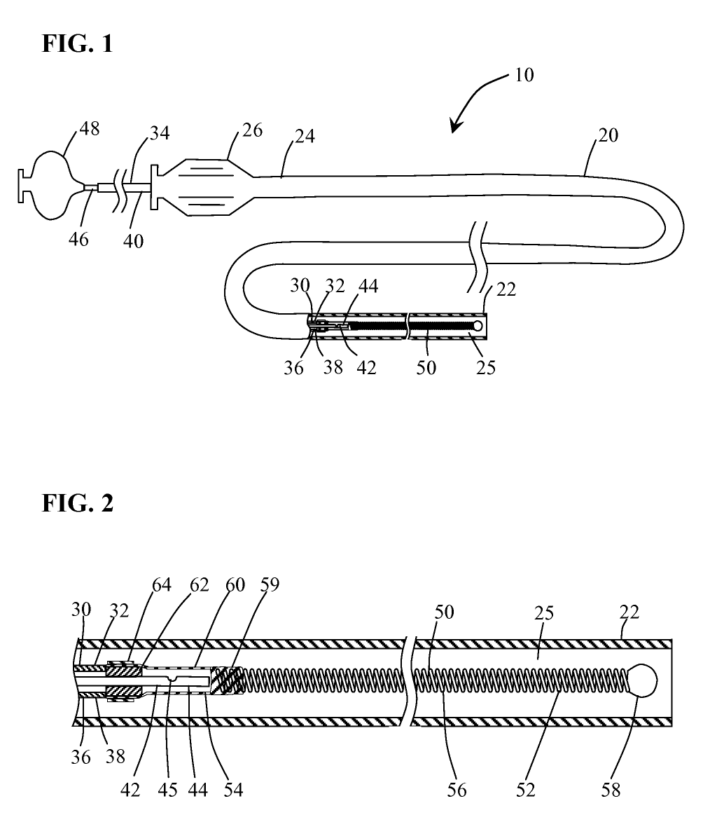

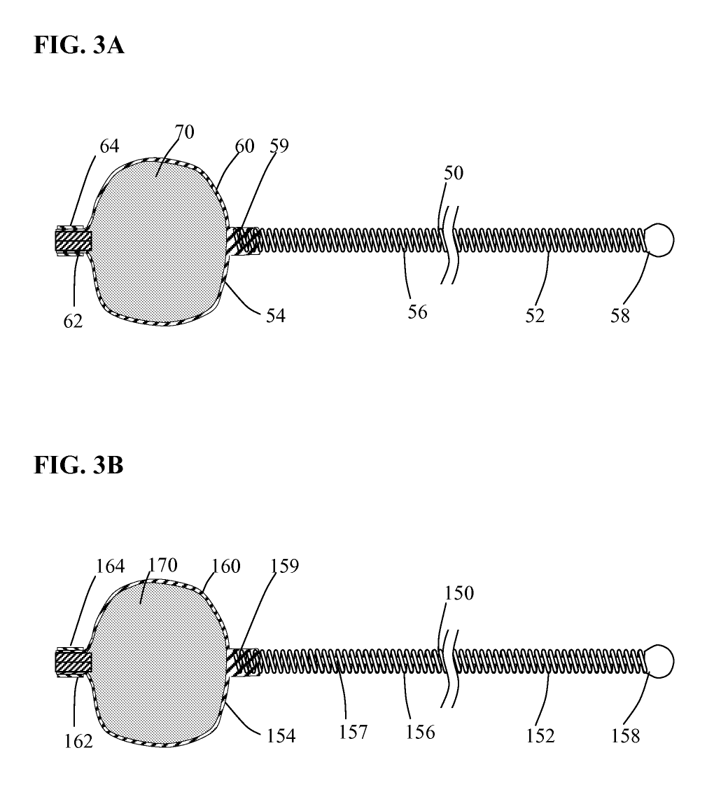

Generally a medical implant deployment system of the present invention may be used to position an implant at a preselected site within the body of a mammal. FIG. 1 generally illustrates embolization system 10 of the present invention which includes elongate catheter 20 having distal and proximal ends 22, 24 and lumen 25 extending therethrough. Proximal end 24 includes catheter hub 26 to facilitate access to lumen 25. Additionally hub 26 includes a Luer connector to facilitate connections with accessory devices commonly used in interventional radiological procedures such as, rotating hemostatic valves. While not shown, the construction of catheter 20 may utilize known catheter technologies that incorporate braiding and or coiling using metallic or non-metallic reinforcing filamentous materials to provide high strength while maintaining catheter flexibility. The term “filamentous” as used herein may be used to describe an object a) composed of or containing filaments b) pertaining to or resembling a filament or c) bearing filaments. The aforementioned definition b) pertaining to or resembling a filament is understood to include general observations of filaments having a substantially longer length relative to its diameter. The incorporation of lubricious hydrophilic and or hydrophobic materials on the inner and or outer surface of the catheter and the application of tip markers are considered to be within the scope of known catheter construction techniques and suitable for uses herein described. Delivery system 30 having distal and proximal ends 32, 34 includes an outer tubular positioning member 36 having distal and proximal ends 38, 40 and an inner tubular filling member 42 having distal end 44, aperture 45 and proximal end 46. Filling member 42 includes hub 48 coupled to proximal end 46 to facilitate coupling to syringes or other fluid delivery sources. Delivery system 30 is positioned within lumen 25 of catheter 20 such that proximal end 34 extends proximal to catheter hub 26. FIG. 2 depicts embolization device 50, having distal and proximal portions 52, 54, which is coupled to delivery system distal end 32 in a removable fashion. Embolization device distal portion 52 includes an elongate filamentous scaffold member that takes the form of elongate embolic coil 56 having atraumatic distal end 58. Proximal portion 54 of embolization device 50 includes joint member 59 which couples the proximal end of embolic coil 56 to expandable balloon member 60. Expandable balloon member 60 includes a proximal sealing valve 62 and a tubular retaining element 64 positioned around valve 62. Distal end 44 of filing member 42 is positioned through sealing valve 62 such that aperture 45 is in fluid communication with balloon member 60. Sealing valve 62 is formed of a resilient material and has a normally closed configuration such that when filling member distal end 44 is withdrawn from sealing valve 12 the sealing valve closes. The resiliency of sealing valve 62 provides a frictional engagement between the valve and distal end 44 of filling member 42. Retaining element 64 preferably takes the form of a radiopaque shrink tubing or marker band to provide visibility under fluoroscopy of the proximal end of embolization device 50 and to restrict the expansion of sealing valve 62 thus providing increased frictional engagement between the valve and filling member distal end 44. Distal end 38 of pusher member 36 is positioned adjacent sealing valve 62 and retaining element 64. Positioning member 36 is preferably formed of a thin walled metallic hypotube however catheter construction materials and techniques may also be suitable. Preferably, distal end 38 of pusher member 36 is flexible but resists axial elongation and compression and has an outer diameter close to the diameter of sealing valve 62. Filling member 42 is also preferably formed of a thin walled metallic hypotube however catheter construction materials and techniques may also be suitable.

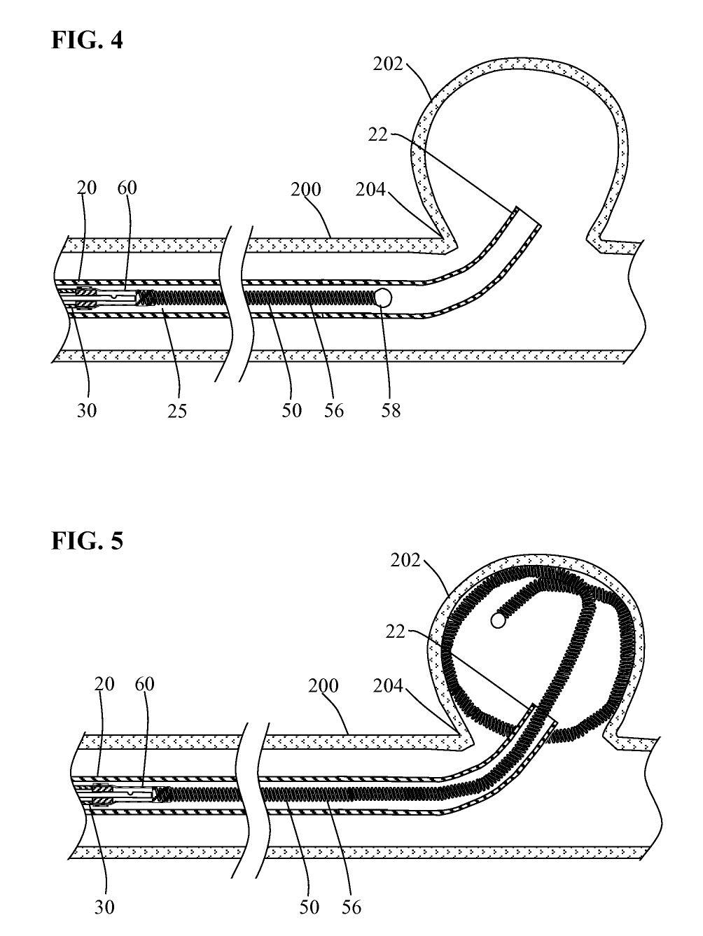

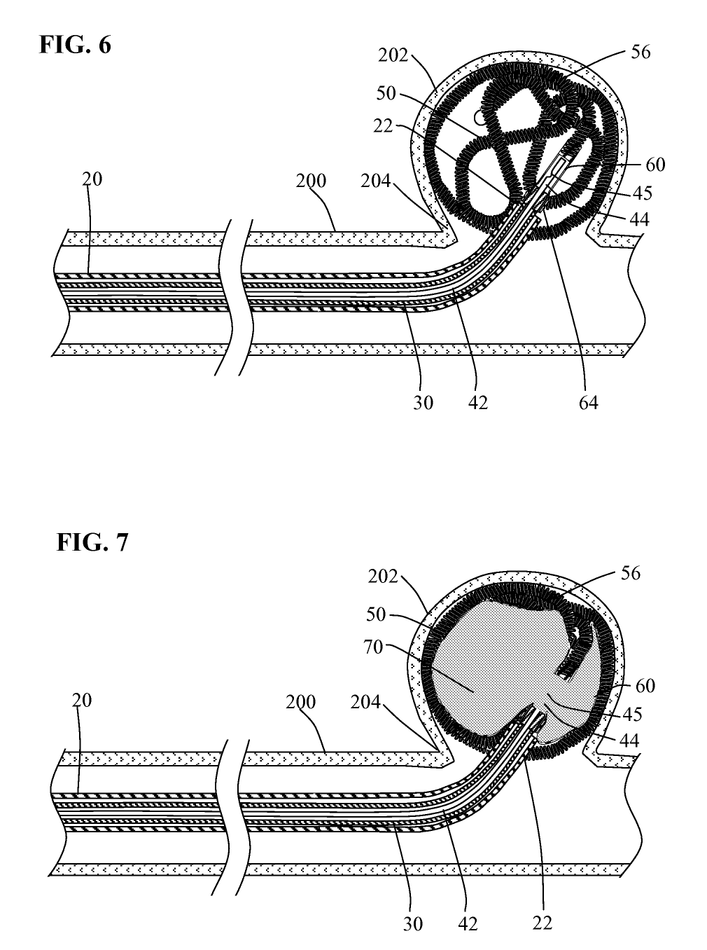

FIG. 3A illustrates embolization device 50 where balloon member 60 has been expanded and sealing valve 62 is closed. Embolic coil 56 of embolization device 50 is typically formed from a helically coiled wire using suitable biocompatible materials such as platinum, nitinol, gold or stainless steel with platinum being a preferred material. The wire depicted in embolic coil 56 has a preferred cross-sectional geometry which is circular although other shapes such as “D”, rectangular and star are also contemplated. Scaffold members such as embolic coil 56 may take other suitable forms such as elongate braids or multi-filar winds. Embolic coil 56 is shown having a generally straight shape for convenience but preferably has a shape and size suited for a target location. Embolic coil 56 has a “primary” coil diameter that ranges from about 0.005 inches to about 0.050 inches and preferably ranges from about 0.008 inches to about 0.040 inches. The length of embolic coil 56 may vary widely and ranges from about 1 cm to about 150 cm with a preferred range of 2 cm to 80 cm. These coils may be shaped into helices or spheres having a “secondary” coil diameter ranging from about 2 mm to 50 mm. The selection of the dimensions for a particular coil is dependent upon the dimensions and geometry of the target anatomical site. For example, to treat an aneurysm having a 7 mm diameter, the embolic coil 56 may preferably have a primary coil diameter in the range of 0.010 inches to 0.020 inches and a shape that is helical or generally spherical with a secondary diameter of about 7 mm to 8 mm dependent upon the stiffness of the coil. These coils may include modifications such as the addition of stretch resistance members to aid in delivery, surface texturing and or the addition of bioactive materials and therapeutic compounds as components or coatings to promote the healing response. Other shapes such as spirals and “hour glasses” may be suitable for other lumenal locations within the body. An alternative embodiment of an embolization device 150 is shown in FIG. 3B where embolic coil 156 includes an elongate shaping wire 157 positioned within the lumen of coil 156. The elongate shaping wire 157 is preferably formed of a resilient material such as nitinol and aids the coil in taking a shape. The shaping wire 157 may be free floating within the lumen of coil 156 or secured at various locations to provide increased stretch resistance.

Balloon member 60, shown in an expanded configuration (FIG. 3A), is preferably formed of an elastomeric material such as silicone although non-elastomeric materials may be suitable for some applications. Suitable materials include other polymeric elastomers such as urethanes, polyether block amide (PEBAX) and synthetic rubbers including polyisoprene, nitrile, chloroprene, ethylene propylene diene rubber as well as non-elastomeric materials such as nylons, polyolefins, polytetrafluoroethylene (PTFE) and polyethylene terephthalate (PET). Balloon member 60 is preferably inflated with a low viscosity fluid 70. Radiopaque fluids such as iodinated contrast solutions may also be suitable and provide the advantage of visibility during inflation. Balloon member 60 may also be inflated using radio-opacified fluids that transition from a liquid to a solid including polymerizable or cross linkable solutions such as alginates, cyanoacrylates and monomers of hydroxyl-ethyl methacrylate (HEMA).

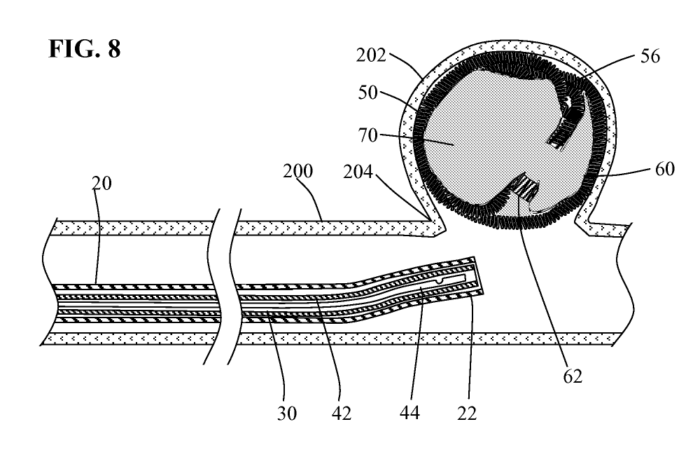

FIGS. 4 through 8 illustrate the method steps of using embolization system 10 to treat an aneurysm of a blood vessel. Embolization system 10 is inserted into blood vessel 200 and catheter 20 is moved to a position within vessel 200 where catheter distal end 22 is positioned within aneurysm 202 adjacent to aneurysm neck 204 (FIG. 4). Delivery system 30, coupled to embolization device 50, is advanced distally within catheter 20 such that embolic coil 56 begins to exit catheter lumen 25 and enter aneurysm 202. Since one of the important purposes of the scaffold is to maintain the stable placement of balloon member 60, the length of embolic coil 56 must be such as to form a stable scaffold. To reliably form a stable scaffold suitable for a wide range of aneurysms embolic coil 56 should have a length of at least three times the inflated diameter of balloon member 60. It is preferable that the length of embolic coil 56 be five to ten times the inflated diameter of balloon member 60 and in some instance more preferable that the length be greater than ten times the inflated diameter of balloon member 60. Typically embolic coil 56 is soft enough to fold upon itself while being delivered into the aneurysm. Further advancement of delivery system 30 allows embolic coil 56 to take a shape within aneurysm 202 forming a scaffold or framework. During delivery, the physician may retract and advance delivery system 30 to reposition embolic coil 56 into the desired scaffold geometry. Once embolic coil 56 is properly positioned, delivery system 30 is advanced to position balloon member 60 within aneurysm 202, distal to catheter distal end 22. A fluid delivery source, such as a fluid filled syringe, is then coupled to filling member hub 48 (not shown). Fluid 70 is delivered to balloon member 60 via filling member 42 to inflate balloon member 60 to a desired volume. It is preferable that fluid 70 is a radiopaque polymerizable liquid, so that the volume filling of balloon member 60 is readily identifiable under fluoroscopy. Upon achieving the desired filling of balloon member 60, filling member 42 is retracted relative to pusher member 36, withdrawing filling member distal end 44 from balloon member 60 thus uncoupling delivery system 30 from embolization device 50 which allows sealing valve 62 to close and seal. The closed sealing valve 62, maintains the inflation of balloon member 60 and the scaffold created by embolic coil 56 retains balloon member 60 within aneurysm 202. Delivery system 30 may then be removed from catheter 20 and the body. If the volume filling of the aneurysm is determined to be insufficient, the physician may deploy another embolization device into the aneurysm and fill to achieve the desired result, otherwise catheter 20 can be removed.

As is apparent, there are numerous modifications of the preferred embodiment described above which will become readily apparent to one skilled in the art. It should be understood that various modifications including the substitution of elements or components which perform substantially the same function in the same way to achieve substantially the same result may be made by those skilled in the art without departing from the scope of the claims which follow.

That which is claimed is:

1. A medical implant system comprising:
an elongate flexible catheter having proximal and distal ends and a lumen extending therethrough;
a medical implant having an elongate scaffold portion coupled to an expandable portion, said expandable portion being positioned proximal to said scaffold portion and including a balloon member and a valve assembly, said balloon member having a first diameter when deflated and a second diameter when inflated with a fluid; and
an elongate delivery system positioned within the lumen of said catheter including a tubular pusher member having proximal and distal ends and a lumen extending therethrough and a tubular filling member having proximal and distal ends and being positioned within the lumen of said pusher member, said filling member distal end being removably coupled to said medical implant.

2. A medical implant system according to claim 1 wherein said elongate scaffold includes an embolic coil.

3. A medical implant system according to claim 1 wherein said fluid includes a radiopaque fluid.

4. A medical implant system according to claim 1 wherein said fluid comprises a fluid that transitions from a liquid to a solid.

5. A medical implant system according to claim 1 wherein at least some of said scaffold portion is positioned within said balloon member.

6. A medical implant system according to claim 1 wherein said embolization device includes at least one expansion resisting member positioned at a location along the length of said balloon member to restrict the expansion of said balloon member at said location.

7. A medical implant system according to claim 2 wherein said embolic coil includes a shaping member.

8. A medical implant system according to claim 2 wherein said embolic coil includes a stretch resistant member.

9. A medical implant for implantation at a target site in the body comprising:
an inflatable balloon member having a valve assembly, said balloon member having a first diameter during delivery to a target site and second diameter when inflated with a fluid and deployed at a target site, wherein said second diameter is greater than said first diameter; and,
an elongate filamentous scaffold member capable of folding upon itself, fixedly coupled to said balloon member and having a length at least three times said second diameter.

10. A medical implant according to claim 9 wherein said elongate scaffold member comprises an embolic coil.

11. A medical implant according to claim 9 wherein said fluid includes a radiopaque fluid.

12. A medical implant according to claim 9 wherein said fluid comprises a fluid that transitions from a liquid to a solid.

13. A medical implant according to claim 9 wherein said balloon member includes at least one expansion resisting member to restrict the expansion of said balloon member.

14. A medical implant according to claim 10 wherein said embolic coil includes a shaping member.

15. A medical implant according to claim 10 wherein said embolic coil includes a stretch resistant member.

16. A medical implant according to claim 10 wherein at least one of said balloon member and scaffold member comprises a bioactive therapeutic material.

17. A medical implant system comprising:
an elongate flexible catheter having proximal and distal ends and a lumen extending therethrough;
a medical implant having an elongate filamentous scaffold portion capable of folding upon itself and an inflatable portion comprising a balloon member and a valve assembly, said scaffold portion being fixedly coupled to said inflatable portion; and
an elongate delivery system positioned within the lumen of said catheter including a pusher member having proximal and distal ends and a tubular filling member having proximal and distal ends wherein said filling member is adapted to deliver a fluid to inflate said inflatable portion and said filling member distal end is removably coupled to said medical implant.

18. A medical implant system according to claim 17 wherein said elongate scaffold includes an embolic coil.

19. A medical implant system according to claim 17 wherein said fluid includes a radiopaque fluid.

20. A medical implant system according to claim 18 wherein said fluid comprises a fluid that transitions from a liquid to a solid.
